(19) United States
(12) Patent Application Publication
(10) Pub. No.: US 2021/0069059 A1
(43) Pub. Date: Mar. 11, 2021

(54) Electroacupuncture Treatment System and Data Optimization Method Thereof

(71) Applicant: Hao Zhou, Shanghai (CN)

(72) Inventor: Hao Zhou, Shanghai (CN)

(21) Appl. No.: 16/747,400

(22) Filed: Jan. 20, 2020

(30) Foreign Application Priority Data
Sep. 10, 2019 (CN) 201910853325.7

(51) Int. Cl. A61H 39/00; G16H 40/40; G16H 20/40

(52) U.S. Cl. CPC A61H 39/002; A61H 2201/501; G16H 20/40; G16H 40/40

(57) ABSTRACT

The invention discloses an electro-acupuncture treatment system and a data optimization method thereof. The embodiments relate to the technical field of traditional Chinese medicine diagnosis and treatment equipment. The patient's pre-treatment evaluation data information is entered through a patient-end device, based on an AI algorithm program of an evaluation device and/or an intelligent computing device. Through further inquiries or diagnostic information, and the pre-treatment evaluation data based on the input data of the patient-side equipment may be continuously improved. Finally, the historical data information in the database may be combined to generate diagnostic information suitable for the user and use the stimulation wave to customize the equipment to transform it into the treatment plan information which may be sent to the patients device after confirmation by doctors and other professionals. Then the stimulation device may act on the human body. The above process may continuously optimize the data, and finally generate different treatment plan data for different individuals.

### RELATED PATENT APPLICATIONS

This application claims priority date of a Chinese Patent application, which was filed on Sep. 10, 2019 with application number 201910853325.7, which is incorporated by reference herein in its entirety.

### TECHNICAL FIELD

This disclosure relates to the technical field of traditional Chinese medicine diagnosis and treatment equipment, and more particularly to an electro-acupuncture treatment system and a data optimization method thereof.

### BACKGROUND

Acupuncture originated in China is now widely used throughout the world. Traditional Chinese medicine believes that acupuncture can stimulate pain relief and discomfort. On Oct. 24, 2018, the US President signed the H.R.6105 which stated that acupuncture could be used for the treatment of pain.

In addition to inserting into the body, doctors use an electroacupuncture needle connected to an electrode plate to stimulate the body with varying electric currents, which is better than a fixed-frequency current stimulus. In recent years, electroacupuncture has become an increasingly popular means of enhancing analgesic effects and suppressing anxiety in the case of various pains.

However, due to the individual difference in each person's body, different human bodies are suitable for electrical stimulation of different frequencies and different intensities. Continuously obtaining accurate data in electroacupuncture treatment is very important for evidence-based medical research and electroacupuncture treatment.

### SUMMARY

During the electroacupuncture treatment, different individuals are suitable for electrical stimulation of different frequencies and intensities. In the current technology, the electroacupuncture treatment scheme is fixed and single, which reduces the therapeutic effect and may even cause side effects. Therefore, the first objection of the present invention is to provide an electroacupuncture treatment system capable of continuously optimizing and adjusting a treatment plan according to individual differences, such as adjusting the frequency and intensity parameters of electro-acupuncture electrical stimulation, and finally confirming that it is most suitable for an individual patient. The treatment plan improves the effect of electroacupuncture treatment. Based on the electroacupuncture treatment system, the second objection of the present invention is to provide a data optimization method for an electroacupuncture treatment system, which can take the input of big data and individual patients and managers as an optimization condition, and finally confirm the optimal solution parameter power supply needle. The treatment system is used, and the specific scheme is as follows:

An electroacupuncture treatment system, comprising a network connected patient device, an evaluation device, an intelligent computing device, a stimulation wave customization device, a stimulation device, a management device, and database. The network connected patient-side device may be used for collecting and transmitting evaluation data information before and after the user's treatment. The data may be sent to the evaluation device. The network connected patient-side device may receive and display the inquiry information output by the evaluation device; The network connected patient-side device may receive the treatment of the stimulation wave customized device and/or the management device output program information, authorized by the user or directly sent to the stimulation device.

The evaluation device, through the network connected patient device, prompts to collect and/or receive the evaluation data information output by the patient device, and generates corresponding inquiry information based on the algorithm program configured in itself and/or the intelligent computing device and sends the corresponding inquiry information to the patient device.

An intelligent computing device may receive evaluation data information from the patient device and/or the evaluation device, generate inquiry information or diagnostic information based on a built-in setting algorithm program, and the inquiry information is output to the patient device, and the diagnosis information may be directly output to the stimulation wave custom device after confirmation by the management terminal;

Stimulating the wave customization device may receive the diagnostic information output by the intelligent computing device, generate corresponding treatment plan information, output to the management device or confirm by the management device and outputting to the patient device;

Stimulating the device may receive the treatment plan information transmitted by the patient device and confirm the treatment plan information according to the setting algorithm, and convert the treatment plan information into a stimulation action acting on the human body;

The management device may receive the treatment plan information output by the stimulation wave custom device, determine the feasibility of the treatment plan included in the treatment plan information according to the operation of the manager, and may output the treatment plan information to the patient device or output confirmation information to the stimulus wave custom device if feasible.

The database may receive and store various data information output by each device and various setting algorithm data.

Through the above technical solution, the user may fully evaluate the condition before the user performs the electroacupuncture treatment, and then the intelligent computing device and the evaluation device according to the pre-treatment evaluation data information and/or the historical evaluation data information.

Generating treatment plan information suitable for the above-mentioned user, and according to the feedback of the patient-side device, the above-mentioned treatment plan information may be continuously optimized in subsequent treatment or data processing, and finally a set of targeted products suitable for the user is generated. The treatment plan greatly enhances the therapeutic effect of the electroacupuncture treatment system.

Further, the patient device, the evaluation device, the management device, and the stimulation wave customization device may be configured as a tablet, a computer, and a mobile smart device having an application interface for interacting with the user.

The application interface is configured to interact with other functional components and external devices built into the corresponding device.

Through the above technical solution, the limitation of the environment to the use of the above system can be reduced, and the user can use it at home or elsewhere, which is simple and convenient.

Further, the patient device has an initialization program module configured to be connected to an external network data to obtain an interaction interface for inputting initial data information.

Through the above technical solution, it is convenient for the user to register or log in, and enter the initial data information.

Further, the patient device is configured with a sensor for identifying patient identity information and an identity program module.

Through the above technical solutions, the safety of the system can be improved, and the use of the above system to interfere with the treatment can be avoided.

Further, a guiding program module for guiding the user to use the evaluation device may be built in the patient end device.

Through the above technical solutions, the user of the patient device can quickly learn to use the evaluation device, thereby reducing the difficulty of using the system.

Additionally, the database may include relational data storage area for storing manually input relationship data; evaluating a data information storage area for storing evaluation data information from the patient end device and/or the evaluation device; an algorithm program storage area for storing related intelligent algorithm program modules; a treatment data information storage area for storing treatment data information of each user.

The system may run a data storage area for storing operational data information of the system.

Further, the network may be configured as a local area network (LAN), a wide area network (WAN), a storage area network, or a combination thereof, and the inter-network communication method is configured as WiFi communication, ethernet communication, bluetooth communication, and optical fiber communication.

The patient device may transmit data to other devices in an encrypted format over the network.

Through the above technical solution, the authenticity and privacy of the input data of the patient device can be ensured.

A data optimization method for an electroacupuncture treatment system may comprise establishing a network connection between devices. The user evaluates the data information before or after the treatment based on the user equipment collected by the patient device.

Generating diagnostic information may be based on an evaluation program built into the evaluation device and/or the intelligent computing device.

The stimulation wave custom device may receive the above diagnostic information, and generate corresponding treatment plan information according to the built-in setting algorithm. The stimulation wave custom device may select whether to transmit the above treatment plan information to the patient device according to the confirmation result of the treatment plan corresponding to the treatment plan information by the management device. After the stimulation device receives the treatment plan information transmitted by the patient device and confirms by the patient device, the treatment plan information is converted into a stimulation action acting on the human body according to the setting algorithm.

Based on the use's post-treatment evaluation data information collected by the patient device, new inquiry information or diagnostic information may be generated according to an evaluation program built in the evaluation device and/or the intelligent computing device.

Through the above technical solutions, it is possible to continuously optimize the treatment plan information for the patient and improve the accuracy of the later data analysis.

Further, the user's pre-treatment or post-treatment evaluation data information collected by the patient-side device may include an evaluation device that receives the pre-treatment evaluation data information output by the patient device, and determines whether further information needs to be supplemented based on an algorithm program configured in itself and/or the intelligent computing device, and if necessary, generates corresponding inquiry information and sends the corresponding inquiry information to the patient device;

The patient terminal device receives the above inquiry information, and outputs the perfect pre-treatment evaluation data information according to the user's operation.

Through the above technical solution, the user can continuously improve the pre-treatment evaluation data information entered by the user in a guided manner, which is helpful for evaluating the analysis of the data by the device and/or the intelligent computing device, so the generated diagnostic information is more accurate.

Further, the generating the diagnostic information according to the algorithm program built in the evaluation device and/or the intelligent computing device may include

A relevant setting algorithm program module in the database and the historical evaluation data information related to the current evaluation data information that are retrieved based on the current evaluation data information, and new diagnostic information may be generated and transmitted to the management device.

Compared with the prior art, the beneficial effects of the present invention are as follows:

The patient's pre-treatment evaluation data information is entered through the patient device, and further inquiry information or diagnostic information is obtained based on the artificial intelligence (AI) algorithm program of the evaluation device and/or the intelligent computing device, and the pre-treatment evaluation data is continuously improved according to the input data of the patient device. Finally, combined with the historical data information in the database, diagnostic information may be generated that is suitable for the user and convert it into treatment plan information, which is confirmed by a doctor or the like and then sent to the patient device, and then the stimulation device is applied to the human body, and the above process can be achieved for continuous optimization of data, and ultimately to generate different treatment plan data for different individuals, is of great significance for medical research analysis and post-treatment.

### BRIEF DESCRIPTION OF THE DRAWINGS

FIG. 1 is a system frame diagram of the electro-acupuncture treatment system of the present application;

FIG. 2 is a schematic diagram of a connection relationship and data transmission between various devices according to an embodiment of the present invention;

FIG. 3 is a schematic diagram of a method for data optimization of an electroacupuncture treatment system according to the present invention.

Callout numbers are following:

1. Patient-side equipment; 2. Evaluation equipment; 3. Intelligent computing equipment; 4. Customized stimulation equipment; 5. Stimulation equipment; 6. Management equipment; 7. Database; 8. Network.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

In order to facilitate the description of the solution of the present invention, specific details are listed in the following description in order to explain the solution of the present invention. The embodiments of the present application described below can be applied or incorporated into different components, such as programs, circuits, devices, and systems. The structure and components shown in the block diagram are schematic illustrations of a specific embodiment of the present application. It should be understood that the connections between the components in the figure are not limited to direct connections. In addition, those skilled in the art should realize that the embodiments of the present invention described below can be implemented or embodied in various ways, such as method flow, equipment, and system.

The connections between components or systems in the diagrams are not limited to direct connections. The data between these components can be modified, reformatted, or otherwise changed by intermediary components. In addition, additional or fewer connections can be used. It should also be noted that the term coupling should be understood to include direct connections, indirect connections through one or more intermediary devices, and wireless connections.

Reference to one embodiment, multiple embodiments, or a specific embodiment in this application refers to the specific features, structures, characteristics, or functions described in at least one embodiment of the present invention, and may be included in multiple implementation example. In addition, the appearances of the above terms in different places in the specification do not necessarily refer to the same embodiment.

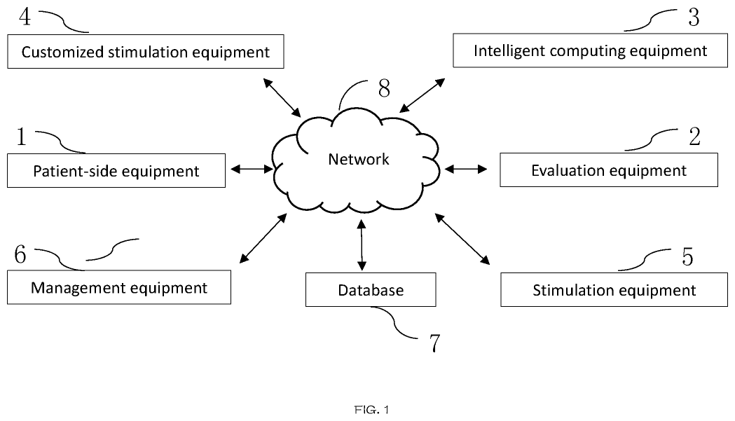

An embodiment of the present application is shown in FIGS. 1 to 3. Specific embodiments of the electroacupuncture treatment system of the present application will be described with various structural and functional components. Those skilled in the art should understand that the above embodiments can be implemented in various environments, including but not limited to hospitals, home care, nursing homes, and rehabilitation centers.

FIG. 1 is a system frame diagram of an embodiment of the electro-acupuncture treatment system of the present application. The above electro-acupuncture treatment system mainly includes a patient-side device 1, an evaluation device 2, an intelligent computing device 3, and a stimulation wave customization device 4, stimulation device 5, management device 6, and database 7.

Among them, the patient-side device 1 is used to collect and input the evaluation data information before and after the treatment of the user and send it to the evaluation device 2; receive and display the inquiry information output by the evaluation device 2; receive the stimulation wave customization device 4 and/or management-side device 6 may be authorized by the user or sent directly to the stimulation device 5.

In the present application, the patient-side device 1, the evaluation device 2, the management-side device 6, and the stimulation wave customization device 4 may be a tablet computer, a computer, a smart phone, or other electronic devices in practice, and are used to interact with the user. Application Programming Interface (API) that can interact with other components within the device, such as touch screens, keyboards, sensors, and even other external devices.

In particular, the patient-side device 1 may also be a self-measurement device or an assistant measurement device that automatically collects or enters the user's pre-treatment evaluation data information, such as an automatic blood pressure meter, an automatic blood glucose meter, or a device that has not been previously trained by a person or the like.

The patient-end device 1 has an initialization program module built therein, and is configured to be used for data connection with an external device to obtain an input interface for inputting initial data information, such as a user's personal name information, age information, and the like.

In some embodiments, the patient-end device 1 may be configured as a smartphone with related applications installed. The applications may be downloaded through a download link provided by a medical service provider such as a hospital. Once the user registers or logs in, the download link may be downloaded. Will be sent to the user's smartphone. Users can then download, install, and execute instructions from the mobile app.

In actual operation, after the patient is registered and authenticated, the PRO data that is the data of the disease and/or the treatment result reported by the user, can be input to the patient-side device 1. Users can use various communication and entry methods, such as entering data in the form of questionnaires sent by mobile phones or using voice to enter data. The patient-end device 1 can communicate the above-mentioned PRO data through a wired communication method or a wireless communication method through the network 8.

In the present application, the network 8 may be a local area network (LAN), a wide area network (WAN), a storage area network (SAN), or a combination thereof. The network 8 may be configured to support various communication methods, including but not limited to WiFi communication, ethernet communication, Bluetooth communication, fiber optic communication, and the like.

In some embodiments, the patient-side device 1 may transmit data to other devices in an encrypted format through the network 8 described above.

In this application, the evaluation device 2 prompts itself through the patient-side device 1 to collect and/or receive the evaluation data information output by the patient-side device 1, and generates corresponding queries based on the algorithm program configured in itself and/or the intelligent computing device 3. The information is sent to the patient-side device 1.

The above-mentioned inquiry information includes a data request and/or a question, and the above-mentioned evaluation device 2 may generate a further request question based on the data received from the patient-side device 1. For example, in practice, the patient may be asked if there is pain in a certain part of the body. If the received data from the patient-end device 1 is a positive answer, then further questions related to pain, such as the level of pain or the pain for specific parts. The above questions are generated and sent to the patient-side device 1.

In this application, the algorithm program applied to the evaluation device 2 may be stored in the evaluation device 2 itself or in the intelligent computing device 3. Generally, for some simple pre-treatment evaluation data information output by the patient-end device 1, the built-in algorithm program in the evaluation device 2 can be used to implement the evaluation and output the corresponding inquiry information. For some complex pre-treatment evaluation data information, the above data information may be transmitted to the intelligent computing device 3 to perform a complex calculation. In a specific embodiment, the above-mentioned intelligent computing device 3 may be configured in the cloud, and the evaluation device 2 performs data communication with the intelligent computing device 3 through the network 8.

The above-mentioned intelligent computing device 3 is internally configured with a set of program algorithms, which usually includes an AI program algorithm, receives evaluation data information from the patient-side device 1 and/or the evaluation device 2, and generates inquiry information based on the built-in set algorithm program or diagnosis information, the inquiry information is output to the patient-side device 1, and the diagnosis information is output to the stimulation wave customization device 4 directly or after being confirmed by the management end.

Further, the above-mentioned inquiry information or diagnosis information can be generated using a program of the intelligent computing device 3 based on artificial intelligence (AI). The artificial intelligence-based algorithm module may include software and/or hardware, which contains an instruction program or a combination of instructions. The generated requests and/or questions are transmitted back to the patient-side device 1 so that the patient can input more data information accordingly. The generated evaluation data information is transmitted to the management device 6 for reference.

In some embodiments, the diagnostic information is generated using an AI program with an evolutionary algorithm built in. The AI program used to generate diagnostic information and the AI program used to generate further inquiry information can share the same hardware, but use different software modules. The intelligent computing device 3 may be a centralized server or a cloud-based server.

In some embodiments, at least one evaluation device 2 corresponds to the patient-side device 1 and/or the management-side device 6. The user is prompted through the patient-side device 1 to use the evaluation device 2 for some self-measurement or auxiliary measurement. The evaluation can be requested by the doctor, and the intelligent computing device 3 generates corresponding inquiry information according to the evaluation data information input by the user and finally sends the inquiry information to the patient-side device 1.

In some embodiments, in order to facilitate the user's familiarity with the operation of the above-mentioned evaluation device 2, the patient-side device 1 has a built-in guidance program module for guiding the user to use the evaluation device 2, that is, a related application can be loaded into the patient-side device 1. Procedures to guide the use of evaluation equipment 2. In some embodiments, it may be configured as multiple application program modules, including program modules with augmented reality functions, to ensure that the user uses the evaluation device 2 correctly. The collected evaluation data can be transmitted from the patient-side device 1 and/or the evaluation device 2 to the intelligent computing device 3 and the management-side device 6 as a reference basis for further operations.

In this application, the stimulus customization device 4 may receive the diagnostic information output from the intelligent computing device 3, generate corresponding treatment plan information, and output the corresponding treatment plan information to the management end device 6 or the patient end device 1 after being confirmed by the management end device 6.

In detail, the stimulation wave customization device 4 receives the evaluation data of the intelligent computing device through the network 8 to customize specific stimulation waves suitable for the patient, including specific pulse parameters, such as stimulation time, frequency, voltage, current, stimulation wave shape, and a combination of the above parameters, etc. The above-mentioned specific pulse parameters are provided to the management end device 6 for confirmation, and after confirmation, they are transmitted to the patient end device 1 to start or end stimulation, and the database 7 saves these data.

In practice, after the stimulation device 5 receives the treatment plan information transmitted by the patient-side device 1 and confirms it, the treatment device information is converted into a stimulation action acting on the human body according to a set algorithm. The above stimulation device 5 may include a program-controlled power generator for outputting a stimulation wave with a set parameter. The output end of the program-controlled power generator is connected with an acupuncture needle through a conductive material, and the conductive material includes, but is not limited to, a wire, a conductive patch, or a conductive clip.

The above-mentioned stimulation wave customizing device 4 is data-connected with the patient-end device 1 through a wired or wireless network 8, and the patient-side device 1 is used to transmit the treatment plan information to the stimulation wave customizing device 4 and control the action of the stimulation wave customizing device 4.

In some embodiments, the patient-end device 1 securely transmits information in an encrypted form to ensure the privacy and authenticity of the user-entered data. The evaluation data collected from the evaluation device 2 is also processed in the same manner. The above-mentioned encrypted communication may be implemented by applying a security function embedded in hardware and/or software of the patient-end device 1, and these hardware and/or software may enable the security function when transmitting and storing sensitive data.

In some embodiments, the patient-end device 1 includes sensors for collecting information related to the identity of the user, which information may be used for identification and/or identity verification of the user. The sensor may be a fingerprint sensor, an iris scanner, or the like. In some embodiments, the sensor may be a camera or a microphone. The patient-end device 1 performs face or voice recognition through a camera or a microphone using pre-loaded software for recognition and/or authentication.

In some embodiments, the patient-side device 1 is a smart phone, a tablet computer, or a notebook computer with an automatic service program installed. The automatic service program can be downloaded through a download link provided by a medical service provider such as a hospital. Provided to the user during registration. After the installation is completed, an application program interface (API) is displayed on the screen of the patient-side device 1 to guide the patient to start the automatic service. The above-mentioned automatic service includes, but is not limited to, the detection data of various instruments used to detect the user's body parameters are automatically acquired and sent to the evaluation device 2.

In this application, the management end device 6 receives the treatment plan information output from the stimulation wave customization device 4 and confirms the feasibility of the treatment plan contained in the above treatment plan information according to the manager's operation judgment. If feasible, outputs the treatment plan information to the patient. The terminal device 1 may output confirmation information to the stimulation wave customization device 4. The management device 6 is preferably configured to a doctor or a caregiver with relevant medical experience.

The database 7 receives and stores various data information and various setting algorithm data output by each device.

In detail, the database 7 mainly includes: a relational data storage area for storing manually entered relational data, and the relational data includes user ID and its corresponding relational data with information such as evaluation data information, diagnosis information, and treatment plan information. Evaluation data information storage area for storing evaluation data information from patient-side device 1 and/or evaluation device 2; algorithm program storage area for storing related intelligent algorithm program modules; treatment for storing treatment data information of each user data information storage area; a system operation data storage area for storing operation data information of the system.

In the above embodiments, there are mainly three data sources in the database 7. The first source is data entered manually, the second source is data from the stimulus customization device 4, and the third source is data from system usage and machine learning. The aforementioned devices are coupled via one or more networks 8 in a wired or wireless connection for information exchange.

It should be noted that each of the above devices can be configured to contain various functional components, and some devices can be integrated as one device. For example, integrating the user-end device and the evaluation device 2 in a smart mobile terminal, you can install application software or apps on these devices to achieve the required functions.

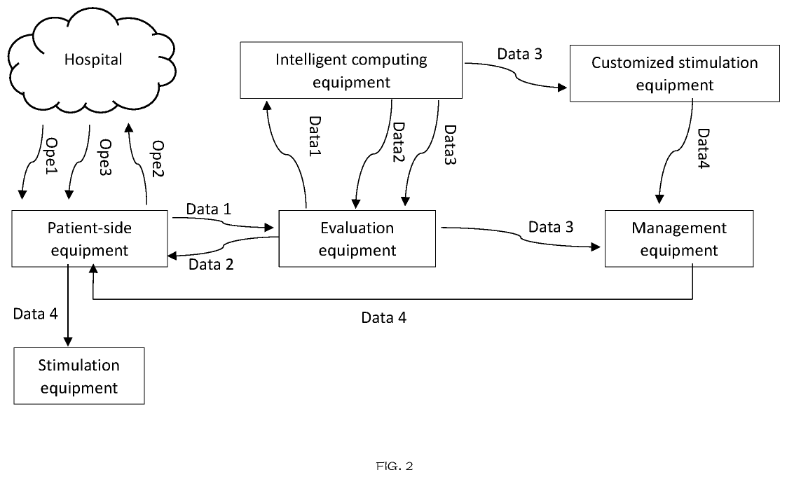

FIG. 2 shows the data connection and transmission relationship between various devices. In FIG. 2, Ope1 indicates that the patient-side device is downloaded through a download link provided by a medical service provider such as a hospital, and Ope2 indicates that the patient-side device performs registration or entry. Ope3 indicates downloading of PRO data. Data1 represents evaluation data information before treatment, Data2 represents inquiry information, Data3 represents diagnostic information, and Data4 represents treatment plan information.

Based on the above electroacupuncture treatment system, the present invention also proposes a data optimization method for the electroacupuncture treatment system. Before describing the above method steps, those skilled in the art should realize that:

The following steps can be performed selectively;

The following steps are not limited to the specific order specified herein;

The following steps can be performed in different orders;

The following steps can be performed simultaneously.

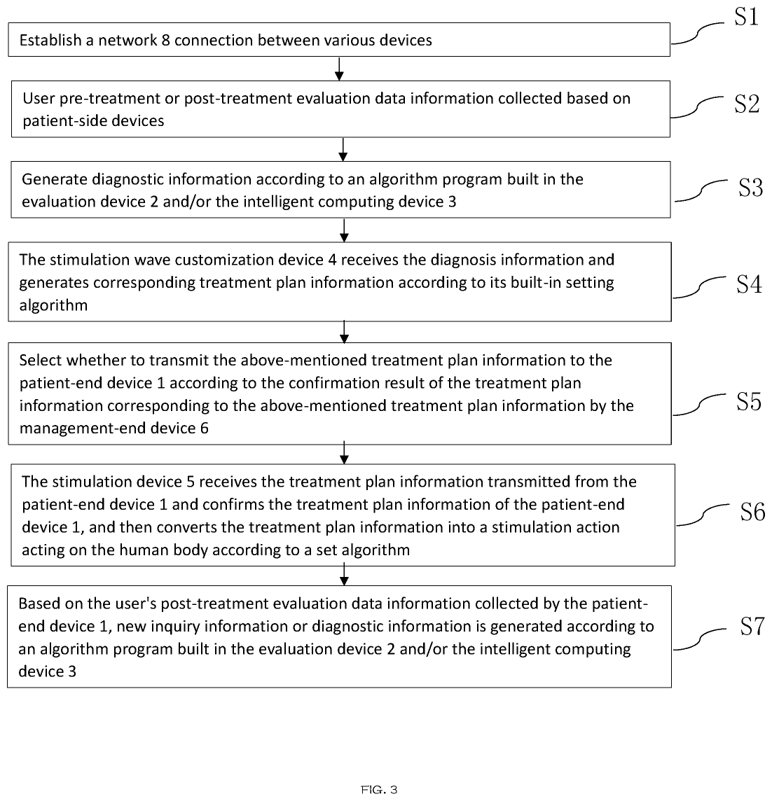

In this application, as shown in FIG. 3, the data optimization method of the electroacupuncture treatment system mainly includes:

S1. Establish a network 8 connection between various devices;

S2. Based on the uses pre-treatment or post-treatment evaluation data collected by the patient-end device 1;

S3. Generate diagnostic information according to an algorithm program built in the evaluation device 2 and/or the intelligent computing device 3.

S4. The stimulation wave customization device 4 receives the diagnosis information and generates corresponding treatment plan information according to its built-in setting algorithm.

S5. Select whether to transmit the above-mentioned treatment plan information to the patient-end device 1 according to the confirmation result of the treatment plan information corresponding to the above-mentioned treatment plan information by the management-end device 6;

S6. The stimulation device 5 receives the treatment plan information transmitted from the patient-end device 1 and confirms the treatment plan information of the patient-end device 1, and then converts the treatment plan information into a stimulation action acting on the human body according to a set algorithm;

S7. Based on the users post-treatment evaluation data information collected by the patient-end device 1, new inquiry information or diagnostic information is generated according to an algorithm program built in the evaluation device 2 and/or the intelligent computing device 3.

After the above step S7 is performed, the steps for subsequent treatment execution may start from step S3. Of course, the patient-side device 1 may also start from the above step S2 and continue to supplement relevant data.

In the above step S1, a network 8 connection between various devices is established. The network 8 may be a local area network (LAN), a wide area network (WAN), a storage area network (SAN), or a combination thereof. The data communication method may be flexibly adopted as required. The description mode includes, but is not limited to, WiFi communication, Ethernet communication, Bluetooth communication, and optical fiber communication. It should be noted that the connection of the network 8 between the devices does not need to be complete in the initial state. For example, when the intelligent computing device 3 is needed, the evaluation device 2 and the intelligent computing device 3 are connected. Of course, it can also be fully connected at the beginning.

The above step S2, based on the users pre-treatment or post-treatment evaluation data information collected by the patient-end device 1, includes:

S21, the evaluation device 2 receives the pre-treatment evaluation data information output from the patient-end device 1, and determines whether further supplementary information is needed based on the algorithm program configured in itself and/or the intelligent computing device 3. If necessary, it generates corresponding inquiry information and Sent to patient-end device 1;

S22. The patient-end device 1 receives the above-mentioned inquiry information, and outputs the completed pre-treatment evaluation data information according to a user operation.

The above steps enable the patient-side device 1 to continuously improve the pre-treatment evaluation data information according to the prompts, so that the diagnostic information generated by the later-stage intelligent computing device 3 is more accurate, and the generated treatment plan information is more suitable for the patient. Similarly, the intelligent computing device 3 can also use the built-in machine learning software to further improve the built-in algorithm based on the data entered by the patient-side device 1 to facilitate more targeted generation of inquiry information in the later stage.

The above step S3, according to an algorithm program built in the evaluation device 2 and/or the intelligent computing device 3, generates diagnostic information, including:

S31: Based on the current evaluation data information, the relevant setting algorithm program module in the database 7 and the historical evaluation data information related to the current evaluation data information are generated, and new diagnostic information is generated and transmitted to the management end device 6. Through the above scheme, the intelligent computing device 3 can refer to historical data information in the database 7 when generating corresponding diagnostic information, thereby making the most accurate diagnosis.

In step S4, the stimulation wave customizing device 4 receives the above-mentioned diagnosis information, and generates corresponding treatment plan information according to its built-in setting algorithm. In practice, the above-mentioned diagnosis information can be expressed by a user or a manager (doctor, nurse, etc.) Cognitive forms, such as text descriptions, but the above information forms cannot be directly applied to the human body. At this time, the above diagnosis information needs to be converted into treatment plan information. As mentioned earlier, the above treatment plan information is expressed as stimulus. The time duration, frequency, waveform, and the like of the wave can be recognized by the stimulation device 5 and the manager.

It should be understood that, before performing the above step S2, in order to obtain the related interactive interface, the patient-side device 1 may download the related application program through a download link provided by a medical service provider such as a hospital, and then register and log in.

The method for data optimization of the electroacupuncture treatment system of the present invention can be summarized as follows: a network 8 connection between various devices is established, and a patient-side device 1 such as a smartphone downloads an interactive interface program through a download link provided by a hospital or a related nursing institution, and the above-mentioned interactive interface The program can be connected to other external devices. The user enters his own information according to the prompt, including personal information, personal disease information, physical condition description information, etc. The above information is called pre-treatment evaluation data information, and the above-mentioned pre-treatment evaluation data information is sent to the evaluation device 2 and the evaluation device 2 processes the pre-treatment evaluation information based on its own built-in setting algorithm program and/or the algorithm program of the intelligent computing device 3, and if the pre-treatment evaluation data information is complete, generate diagnostic information based on the pre-treatment evaluation data information, If the above-mentioned pre-treatment evaluation data information is not complete, the corresponding inquiry information is generated and transmitted to the patient-end device 1, and the user continues to improve the related evaluation data according to the prompts until the diagnosis information is generated. The data in the above interaction process is stored in the database 7.

The diagnostic information generated above is sent to the management-end device 6 and the patient-end device 1 for reference, and sent to the stimulation wave customization device to generate corresponding treatment plan information, that is, the duration, frequency, and waveform of the stimulation wave on the electroacupuncture. The above treatment plan information is sent to the management-end device 6 for confirmation. If the management end thinks that the above-mentioned treatment plan is feasible, the above-mentioned treatment plan information is sent to the patient-side device 1. After referring to the above-mentioned treatment plan information, the user can selectively control the stimulation device 5 to execute the treatment plan corresponding to the above treatment plan information to achieve treatment; and then send the evaluation data information after the treatment to the evaluation device 2. Similarly, the post-treatment evaluation data information sent by the patient-side device 1 can be further generated here. The information is inquired and sent to the patient-side device 1 until the evaluation data information is improved after the treatment, and new diagnostic information is generated. The database 7 stores data information in the above process.

The above new diagnostic information can be used as a reference when data analysis is performed later or next diagnosis, so that the system data can be continuously optimized.

The above description is only the preferred embodiments of the present invention, and the protection scope of the present invention is not limited to the above embodiments, and any technical solution belonging to the idea of the present invention belongs to the protection scope of the present invention. It should be noted that, for those of ordinary skill in the art, without departing from the principles of the present invention, several improvements and retouches should be considered as the protection scope of the present invention.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed is:

1. An electroacupuncture treatment system, comprising a patient-side device, an evaluation device, an intelligent computing device, a stimulation wave customization device, and a patient device connected via a communication network, stimulation device, management device, and database;
wherein the patient-side equipment is for collecting and sending evaluation data information before and after treatment to the user and sending it to the evaluation device; receiving and displaying inquiry information output by the evaluation device; receiving stimulation wave customized equipment or the treatment plan information output by the management device, authorized by the user or directly sent to the stimulation device;
wherein the evaluation device is prompted by the patient-side device to collect or receive the evaluation data information output by the patient-side device, and is generated based on the algorithm program configured in itself or the intelligent computing device. The corresponding inquiry information is sent to the patient-side device;
wherein the intelligent computing device receives the evaluation data information from the patient-side device or the evaluation device, and generates inquiry information or diagnosis information based on a built-in setting algorithm program, and the inquiry information is output to patient-side equipment, the diagnostic information is output directly or after being confirmed by the management terminal to the stimulation wave customized equipment;
wherein the stimulation wave customized device receives the diagnostic information output from the intelligent computing device, generates corresponding treatment plan information, and outputs the information to the management terminal device or the patient after being confirmed by the management terminal device;
wherein the stimulation device receives the treatment plan information transmitted by the patient-side device and confirms the patient-side device, and then converts the treatment plan information into a stimulation action acting on the human body according to a set algorithm;
wherein the management device receives the treatment plan information output from the stimulation wave customized device, and confirms the feasibility of the treatment plan contained in the above treatment plan information according to the manager's operation judgment. If feasible, outputs the treatment plan information to Patient-side equipment or output confirmation information to stimulation wave custom equipment;
the database receives and stores various data information and various setting algorithm data output by each device.

2. The electro-acupuncture treatment system according to claim 1, wherein the patient-side device, the evaluation device, the management-end device, and the stimulation wave customization device are configured to have tablet computers, computers, mobile smart devices with application program interfaces that interact with users, the application program interface is configured to interact with other functional components built into the corresponding device and external devices.

3. The electroacupuncture treatment system according to claim 2, characterized in that the patient-side device has a built-in initialization program module configured to be used for data connection with an external device to obtain an interactive interface for entering initial data information.

4. The electro-acupuncture treatment system according to claim 1, wherein the patient-side device is configured with a sensor for identifying patient identification information and an identification program module.

5. The electroacupuncture treatment system according to claim 1, characterized in that the patient-end device has a built-in guidance program module for guiding the user to use the evaluation device.

6. The electroacupuncture treatment system according to claim 1, wherein the database comprises: relational data storage area for storing manually entered relational data; evaluation data information storage area, for storing evaluation data information from the patient-side device or the evaluation device; algorithm program storage area for storing related intelligent algorithm program modules; the treatment data information storage area is used to store the treatment data information of each user; the system operation data storage area is used to store system operation data information.

7. The electro-acupuncture treatment system according to claim 1, wherein: the network is configured as a local area network, a wide area network, a storage area network, or a combination thereof, and the communication method between the networks is configured as WiFi communication, Ethernet communication, Bluetooth communication, and optical fiber communication; the patient-side device transmits data to other devices in an encrypted format through the network.

8. A data optimization method for an electroacupuncture treatment system, comprising: establishing a network connection between various devices; pre-treatment or post-treatment evaluation data information collected by the patient-end device; generating diagnostic information according to an algorithm program built in the evaluation device or the intelligent computing device; the stimulation wave customization device receives the above-mentioned diagnosis information, and generates corresponding treatment plan information according to its built-in setting algorithm; selecting whether to transmit the above-mentioned treatment plan information to the patient-end device according to the confirmation result of the management plan device on the treatment plan corresponding to the above-mentioned treatment plan information; after the stimulation device receives the treatment plan information transmitted from the patient-side device and confirms it, the treatment device information is converted into a stimulation action acting on the human body according to a set algorithm; Based on the user's post-treatment evaluation data information collected by the patient-side device, new inquiry information or diagnostic information is generated according to the algorithm program built in the evaluation device or the intelligent computing device.

9. The method for optimizing data of an electro-acupuncture treatment system according to claim 8, characterized in that the user's pre-treatment or post-treatment evaluation data information based on the patient-end device comprises: the evaluation device receives the pre-treatment evaluation data information output by the patient-end device, and determines whether further supplementary information is needed based on the algorithm program configured in itself or the intelligent computing device, and if necessary, generates a corresponding information and sends it to the patient device; the patient-side equipment receives the above-mentioned inquiry information, and outputs the completed pre-treatment evaluation data information according to the user's operation.

10. The data optimization method for an electroacupuncture treatment system according to claim 8, wherein the generating diagnostic information according to an algorithm program built in the evaluation device or the intelligent computing device comprises: based on the current evaluation data information, the related setting algorithm program module in the database and the historical evaluation data information related to the current evaluation data information are used to generate new diagnostic information and transmit it to the management device.
